(19) United States
(12) Patent Application Publication
(10) Pub. No.: US 2026/0084308 A1
(43) Pub. Date: Mar. 26, 2026

(54) COMPLEX SPATIAL BOUNDARY CONTROL METHOD, SYSTEM, AND ELECTRONIC DEVICE FOR ORTHOPEDIC SURGICAL ROBOT

(71) Applicant: TINAVI MEDICAL TECHNOLOGIES CO., LTD., Beijing (CN)

(72) Inventor: Chongxu LIU, Beijing (CN)

(73) Assignee: TINAVI MEDICAL TECHNOLOGIES CO., LTD., Beijing (CN)

(21) Appl. No.: 19/404,688

(22) Filed: Dec. 1, 2025

Related U.S. Application Data
(63) Continuation of application No. PCT/CN2025/129240, filed on Oct. 22, 2025.

(30) Foreign Application Priority Data
Oct. 31, 2024 (CN) 202411542043.2

(51) Int. Cl. B25J 9/16; A61B 17/16; A61B 34/32; B25J 15/00

(52) U.S. Cl. CPC B25J 9/1679; A61B 17/1615; A61B 34/32; B25J 15/0019; G05B 2219/45117; G05B 2219/45171

(57) ABSTRACT

A complex spatial boundary control method includes: obtaining the target desired position of the center of the ball head of a target grinding drill mounted at the operating end of the robot, complex spatial boundary data, and the radius of the target grinding drill ball head; where the complex spatial boundary data is a closed manifold polyhedron enveloped by triangular facets; and determining whether the target desired position is located outside the complex spatial boundary, and determining, based on the judgment result, whether to correct the target desired position according to the radius of the target grinding drill ball head, to obtain the safe desired position of the ball head center. The method can dynamically compensate for the radius of a high-speed grinding drill ball head, solving the problem that different compensating boundary models must be pre-generated and dynamically managed/switched for grinding tools with different ball head radii.

### CROSS-REFERENCE TO RELATED APPLICATIONS

This application is a continuation of International Patent Application No. PCT/CN2025/129240 with a filing date of Oct. 22, 2025, designating the United States, now pending, and further claims priority to Chinese Patent Application No. 202411542043.2 with a filing date of Oct. 31, 2024. The content of the aforementioned applications, including any intervening amendments thereto, is incorporated herein by reference.

### TECHNICAL FIELD

The present disclosure relates to the field of robotic motion control technology, and particularly to a complex spatial boundary control method, system, and electronic device for an orthopedic surgical robot.

### BACKGROUND ART

The human knee joint is composed of the articular surfaces between the femur, tibia, and patella. It connects the femur and tibia and is one of the joints bearing the greatest load in the body. The knee joint can be divided into the following three compartments: Medial compartment: the medial region of the knee joint, including the medial femoral condyle, tibial plateau, and medial meniscus. Lateral compartment: the lateral region of the knee joint, including the lateral femoral condyle, tibial plateau, and lateral meniscus. Anterior compartment: the anterior region of the knee joint, including the femoral condylar eminence, trochlear groove, patella, and patellar tendon, which play an important role in maintaining knee joint stability and motion. These three compartments collectively form the structure of the knee joint. They cooperate to provide support, movement, and stability. When pathological changes occur in the knee joint, such as severe osteoarthritis, rheumatoid arthritis, or post-traumatic arthritis, one or more compartments may be affected, leading to loss of normal function. In such cases, joint replacement surgery is required, in which the diseased part is replaced with an artificial prosthesis to relieve pain, correct deformity, restore joint function, and improve the patient's quality of life.

Partial knee arthroplasty (PKA) is a surgical method used to treat certain knee disorders. Compared with total knee arthroplasty (TKA), PKA replaces only part of the knee joint, usually one or a combination of two of the medial, lateral, or anterior compartments, and is mainly suitable for patients with localized joint degeneration where the remaining structures are healthy.

Compared with TKA, PKA better preserves healthy bone and soft tissue and retains the knee's natural structure. Patients typically experience more natural joint motion and stability after surgery, while also reducing the use of artificial prostheses. Due to the involvement of fewer tissues in PKA, postoperative pain and discomfort are usually milder, and patients recover faster, enabling them to resume normal life and activities more quickly. PKA also reduces the risk of complications such as infection and thrombosis compared with TKA.

However, PKA has some technical difficulties compared to TKA. PKA requires accurate positioning and grinding of the damaged area of the knee joint (i.e., precise bone surface preparation) to ensure the correct position of the artificial prosthesis and adapt to the patient's physiological anatomy. For doctors, accurately identifying and locating the lesion area is the key to successful surgery. PKA also requires doctors to effectively protect the soft tissue structures around the knee joint, especially ligaments and synovium. During the surgical process, avoiding damage or stretching of these structures is crucial for the patient's postoperative recovery and functional reconstruction. In addition, PKA requires doctors to accurately install artificial prostheses onto the damaged knee joint area.

In traditional PKA, doctors typically use various surgical instruments manually to perform bone surface preparation procedures. Specifically, doctors will use surgical instruments such as surgical blades, bone biting forceps, swing saws, and drills to cut, trim, and grind the surface of damaged bones in order to clean the bone surface and implant artificial prostheses. On the one hand, manual operation reduces the positioning accuracy and shape accuracy of bone surface preparation, which may damage the patient's soft tissues and ligaments, and reduce the overall therapeutic effect of the surgery; On the other hand, frequent tool changes and large grinding forces also impose a significant burden on doctors.

Some methods to improve accuracy involve forming files or resection guides. For example: In milling the curved surfaces of the femoral condyles, holes are pre-drilled to control the direction and depth of the forming file (see CN217960230U, Unicondylar grinding and resection system). In trochlear groove preparation, a resection guide plate is fixed to the femur with bone pins, and the surgeon uses handheld oscillating saws and milling cutters for resection. These methods require extra drilling or pinning, complicating the procedure. Moreover, hand-held tools cannot guarantee complete protection of soft tissues and ligaments.

Robotic-assisted partial knee arthroplasty allows the mechanical arm to assist the surgeon in interactive bone surface preparation, where the robot drives a high-speed grinding drill that the surgeon triggers for controlled operation. This enhances accuracy in bone surface preparation, improves surgical outcomes, eliminates frequent tool changes, and reduces physical strain. However, when performing robot-assisted bone surface preparation, the position of the high-speed grinding drill in space must be restricted within a defined boundary to prevent excessive movement that could damage soft tissues and ligaments or reduce accuracy.

Clinically, multiple grinding drill ball heads of different radii are often used in one procedure to achieve different cutting effects. Thus, the system must dynamically compensate for variations in the ball head radius to offset grinding errors. A similar concept—dynamic tool size compensation—is commonly applied in numerical control (NC) machining. This technique dynamically adjusts the position of the tool center point through a control algorithm during machining to compensate for dimensional errors caused by variations in tool size (for example, the radius of a ball-end drill or a milling cutter). In this way, the final machined dimensions of the workpiece accurately match the intended design dimensions. Existing techniques, however, have the following problems:

CN116077135A—“Bone Grinding Implementation Method in Unicondylar Replacement Surgery”: This method achieves grinding of arbitrary shapes by controlling the power supply such that the drill bit of the orthopedic drill gun can rotate only within a predefined grinding region, thereby achieving polishing of any shape. Essentially, however, this method cannot realize strict boundary control in the true sense. It cannot ensure that the movement of the drill bit, driven by the robotic arm, remains strictly within the prescribed range. It can only achieve passive power cutoff after the motion has already exceeded the boundary, and thus cannot reliably ensure patient safety.

CN116712169A—“Motion Control Method, Apparatus, Equipment, and Storage Medium for an End Tool”: This method performs boundary control based on the nearest image pixel points. When image pixels contain noise or abrupt changes, it causes problems of continuity in motion control. Moreover, the pixel-based judgment introduces angular judgment conditions, which in turn require inverse trigonometric function computations, increasing computational complexity. In addition, when determining ray intersections, the method fails to consider cases where the ray intersects boundary lines or vertices, causing floating-point rounding errors that affect the number of calculated ray intersections. Consequently, when the ray intersects with a boundary line or vertex, the judgment of whether a point is inside or outside the boundary becomes abnormal.

CN118106975B—“Robot-Based Complex Spatial Boundary Control Method, Device, and Robot”: This method includes: obtaining the desired position of the robot's operating end; determining, using a point-in-complex-boundary detection algorithm, whether the desired position is located inside the complex spatial boundary. If the desired position is inside the boundary, the safe position is set equal to the desired position, and the robot's operating end is controlled to work at this safe position. If the desired position is outside the boundary, the method uses a fast algorithm for the minimum distance and nearest point from any point in space to a triangular facet, iteratively computing the nearest distance and nearest point from the desired position to each triangular facet that forms the complex spatial boundary. The nearest point corresponding to the smallest minimum distance is then selected as the safe position output, and the robot's operating end is controlled to operate at this safe position.

In all three methods above, the control model only considers the position of the center of the ball head of the high-speed grinding drill, without taking into account dynamic compensation for the ball head radius. In practical surgical scenarios where multiple grinding drill ball heads of different radii are used for fine edge grinding in the same procedure, the software must pre-store multiple inward-offset boundary models corresponding to different ball head radii and generate them offline before surgery. This introduces substantial data preprocessing, software scheduling and management, and other unnecessary tasks, while also creating potential risks.

CN110997247B—“Robotic System and Method for Generating Reaction Forces to Realize Virtual Boundaries”: This patent discloses a polygon-mesh-based virtual boundary control method, which calculates penetration reaction forces between interactive virtual volumes and polygonal elements. The calculated reaction force is applied to the virtual volume to reduce its penetration into the polygonal elements, thereby achieving boundary control. Although this method takes into account the radius of the high-speed grinding drill ball head, its essence is to control the boundary through virtual damping force, i.e., controlling tool position indirectly through force control. In engineering practice, however, this approach suffers from difficulty in determining the damping coefficient and lack of adequate boundary stiffness. If the damping coefficient is set improperly or the applied force is excessive, the control result may exceed the boundary, making it impossible to achieve strict boundary control.

Therefore, the existing technologies still require further improvement.

### SUMMARY OF THE INVENTION

The purpose of the present disclosure is to overcome the deficiencies in the existing technologies described above and to provide a complex spatial boundary control method, system, and electronic device for an orthopedic surgical robot, thereby solving the problems existing in the prior art.

In one aspect, the present disclosure provides a complex spatial boundary control method for an orthopedic surgical robot, comprising:
S100: obtaining a target desired position of a center of a target grinding drill ball head mounted at an operating end of the robot, complex spatial boundary data, and a radius of the target grinding drill ball head; wherein the complex spatial boundary data is a closed manifold polyhedron enveloped by triangular facets;
S200: determining whether the target desired position is located outside the complex spatial boundary to obtain a judgment result, and based on the judgment result, determining whether to correct the target desired position according to the radius of the target grinding drill ball head, to obtain a safe desired position of the center of the target grinding drill ball head;
S300: controlling motion of the operating end of the robot according to the safe desired position.

Specifically, the determining, based on the judgment result, whether to correct the target desired position according to the radius of the target grinding drill ball head to obtain the safe desired position of the ball head center, comprises:
when the target desired position is located inside the complex spatial boundary, calculating a nearest distance from the target desired position to the complex spatial boundary, and determining whether the nearest distance is greater than or equal to the radius of the target grinding drill ball head; based on the determination, deciding whether to correct the target desired position according to the radius of the target grinding drill ball head to obtain the safe desired position of the ball head center, specifically:
when the nearest distance is greater than or equal to the radius of the target grinding drill ball head, directly using the target desired position as the safe desired position of the center of the target grinding drill ball head;
when the nearest distance is less than the radius of the target grinding drill ball head, correcting the target desired position according to the radius of the target grinding drill ball head to obtain the safe desired position of the center of the target grinding drill ball head.

Specifically, the correcting the target desired position according to the radius of the target grinding drill ball head to obtain the safe desired position of the ball head center comprises:
using the target desired position as an initial search position, selecting a nearest point from the initial search position to the complex spatial boundary and defining the nearest point as an initial nearest point; calculating a distance from the initial search position to the initial nearest point as a target distance; calculating a difference between the radius of the target grinding drill ball head and the target distance as a distance to be corrected; taking a direction from the initial search position toward the initial nearest point as an initial search direction, and moving the initial search position along the initial direction by the distance to be corrected to obtain the safe desired position.

Specifically, the determining whether the target desired position is located outside the complex spatial boundary to obtain a judgment result, and based on the judgment result, determining whether to correct the target desired position according to the radius of the target grinding drill ball head to obtain the safe desired position of the ball head center, comprises:
when the target desired position is located on the complex spatial boundary, correcting the target desired position according to the radius of the target grinding drill ball head to obtain the safe desired position of the center of the target grinding drill ball head.

Specifically, the correcting the target desired position according to the radius of the target grinding drill ball head to obtain the safe desired position of the center of the target grinding drill ball head comprises:
using the target desired position as an initial search position, taking the radius of the target grinding drill ball head as a distance to be corrected; taking the initial search position as a foot of a perpendicular, constructing a perpendicular line from the initial search position to a boundary on which the initial search position lies; taking a direction of a ray formed by the foot of the perpendicular and the perpendicular line as an initial search direction; and moving the initial search position along the initial search direction by the distance to be corrected to obtain the safe desired position.

Specifically, the determining whether the target desired position is located outside the complex spatial boundary to obtain a judgment result and, based on the judgment result, determining whether to correct the target desired position according to the radius of the target grinding drill ball head to obtain the safe desired position of the ball head center, comprises:
when the target desired position is located outside the complex spatial boundary, correcting the target desired position according to the radius of the target grinding drill ball head to obtain the safe desired position of the ball head center.

Specifically, the correcting the target desired position according to the radius of the target grinding drill ball head to obtain the safe desired position of the center of the target grinding drill ball head comprises:
selecting a nearest point from the target desired position to the complex spatial boundary and taking the nearest point as an initial search position; taking the radius of the target grinding drill ball head as a distance to be corrected; taking the initial search position as a foot of a perpendicular, constructing a perpendicular line to a boundary on which the initial search position lies; taking a direction of a ray formed by the foot of the perpendicular and the perpendicular line as an initial search direction; and moving the initial search position along the initial search direction by the distance to be corrected to obtain the safe desired position.

Specifically, the moving the initial search position along the initial search direction by the distance to be corrected to obtain the safe desired position further comprises:
after moving the initial search position along the initial search direction by the distance to be corrected, obtaining a position to be verified (candidate desired position); calculating a minimum distance from the position to be verified to the complex spatial boundary; and determining whether the minimum distance is greater than or equal to the radius of the target grinding drill ball head;
when the minimum distance is less than the radius of the target grinding drill ball head, repeating the above correction process on the position to be verified until the corrected position to be verified lies inside the complex spatial boundary and, after calculating the minimum distance from the corrected position to be verified to the complex spatial boundary, the minimum distance is greater than or equal to the radius of the target grinding drill ball head, and taking the corrected position to be verified as the safe desired position; and
when the minimum distance is greater than or equal to the radius of the target grinding drill ball head, directly taking the position to be verified as the safe desired position.

Specifically, the method further comprises:
when the minimum distance from the corrected position to be verified to the complex spatial boundary is greater than or equal to the radius of the target grinding drill ball head, determining whether a difference between the minimum distance and the radius of the target grinding drill ball head is less than or equal to a first preset threshold; when the difference between the minimum distance and the radius of the target grinding drill ball head is less than or equal to a first preset threshold, taking the corrected position to be verified as the safe desired position;
when the difference between the minimum distance and the radius of the target grinding drill ball head is greater than a first preset threshold, repeating the correction process on the position to be verified until the difference between the minimum distance and the radius of the target grinding drill ball head is less than or equal to the first preset threshold, and taking the corrected position to be verified at that time as the safe desired position.

Specifically, the method further comprises:
recording a number of corrections, determining whether the number of corrections is greater than or equal to a second preset threshold, and determining whether a safe desired position has been found; and based on these determinations, deciding whether the correction of the target desired position is abnormal;
when the number of corrections is greater than or equal to the second preset threshold and no safe desired position has been found, determining that the correction of the target desired position is abnormal;
when the number of corrections is less than the second preset threshold and a safe desired position has been found, determining that the correction of the target desired position is normal; and
when the number of corrections is less than the second preset threshold and no safe desired position has yet been found, determining that the correction of the target desired position is normal, and continuing the correction of the target desired position.

Specifically, the method further comprises:
when the number of corrections is greater than or equal to the second preset threshold and no safe desired position has been found, determining whether the difference between the minimum distance from the current position to be verified to the complex spatial boundary and the radius of the target grinding drill ball head is less than or equal to a third preset threshold; when the difference between the minimum distance from the current position to be verified to the complex spatial boundary and the radius of the target grinding drill ball head is less than or equal to a third preset threshold, taking the current position to be verified as the safe desired position; when the difference between the minimum distance from the current position to be verified to the complex spatial boundary and the radius of the target grinding drill ball head is greater than a third preset threshold, repeating the above correction process on the current position to be verified, and re-recording the correction count, until the difference between the minimum distance and the radius of the target grinding drill ball head is less than or equal to the third preset threshold, and taking the corrected position to be verified at that time as the safe desired position.

Specifically, the method further comprises:
determining whether a re-recorded number of corrections is greater than or equal to a fourth preset threshold, and determining whether a safe desired position has been found; and based on the judgment results, determining whether the correction of the target desired position is abnormal;
when the re-recorded number of corrections is greater than or equal to the fourth preset threshold and no safe desired position has been found, determining that the correction of the target desired position is abnormal;
when the re-recorded number of corrections is less than the fourth preset threshold and a safe desired position has been found, determining that the correction of the target desired position is normal; and
when the re-recorded number of corrections is less than the fourth preset threshold and no safe desired position has yet been found, determining that the correction of the target desired position is normal, and continuing the correction of the target desired position.

In another aspect, the present disclosure provides a complex spatial boundary control system for an orthopedic surgical robot, comprising:
an acquisition module, configured to obtain a target desired position of a center of a target grinding drill ball head mounted at the operating end of the robot, complex spatial boundary data, and a radius of the target grinding drill ball head; wherein the complex spatial boundary data is a closed manifold polyhedron enveloped by triangular facets;
and a control module, configured to determine whether the target desired position is located outside the complex spatial boundary to obtain a judgment result, and based on the judgment result, determine whether to correct the target desired position according to the radius of the target grinding drill ball head, to obtain the safe desired position of the center of the target grinding ball head; and to control motion of the operating end of the robot according to the safe desired position.

In yet another aspect, the present disclosure provides an electronic device, comprising: a memory and a processor; wherein the memory stores computer-readable instructions stored in the memory are executed by the processor to implement the complex spatial boundary control method for an orthopedic surgical robot described above.

### Beneficial Effects

1. The present disclosure proposes and designs a specific implementation scheme for complex spatial boundary control, which requires only the desired position of the high-speed grinding drill, the radius of the ball head of the high-speed grinding drill, and the safety boundary. The current position of the high-speed grinding drill does not need to be obtained, thereby reducing the influence of system delay (latency) on control performance, while ensuring that the resulting safe position of the high-speed grinding drill remains consistent with the intended motion direction.

2. The complex spatial boundary control method proposed by the present disclosure can dynamically compensate for the radius of the high-speed grinding drill ball head, thereby resolving the issue in existing methods where grinding tools with different ball head radii require the pre-generation of boundary models of different compensation sizes, along with dynamic management and switching of those models during operation.

3. The complex spatial boundary control method proposed by the present disclosure imposes no restrictions on the convexity or concavity of the boundary surface. It supports the customization of boundary types according to the patient's anatomical structure and the shape of the artificial prosthesis. This enables the protection of the patient from additional collateral injury and avoids unnecessary over-grinding of soft tissue and ligaments.

4. Through the proposed complex spatial boundary control, when a surgeon's manually planned tool position exceeds the boundary, the system can correct that position to a location within the specified boundary range and in the intended direction. This ensures that bone surface preparation is achieved precisely while allowing the artificial prosthesis to be accurately installed without warping or gaps.

5. Compared with traditional bone surface preparation methods that require frequent tool changes and multiple measuring or guiding devices, the method provided by the present disclosure uses a single high-speed grinding drill power tool in combination with ball heads of different radii to perform bone surface preparation. This eliminates the need to replace grinding or cutting power tools, significantly improving surgical efficiency and surgeon comfort, while also shortening the surgery time and accelerating postoperative recovery for the patient.

6. By applying the method proposed by the present disclosure, when doctors perform bone surface preparation operations on patients' bones and need to dynamically adjust the position of high-speed grinding drills within complex spatial boundaries, the robotic arm can define a safe area for doctors to dynamically adjust, protect patients from unnecessary injuries, and ensure the safety and accuracy of bone surface preparation operations.

### DETAILED DESCRIPTION OF THE DRAWINGS

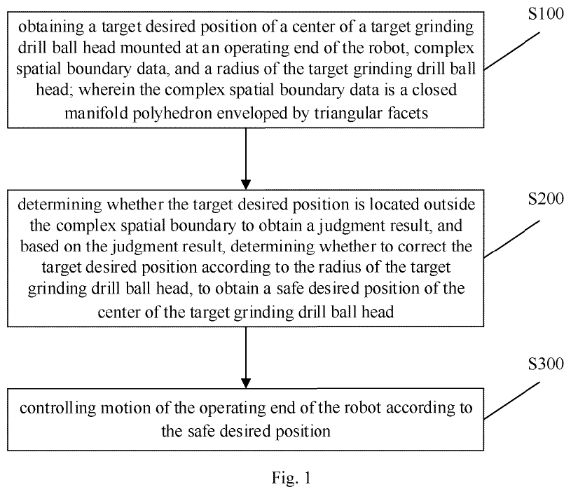

FIG. 1 is a schematic flow diagram of the complex spatial boundary control method for an orthopedic surgical robot according to a specific embodiment of the present disclosure.

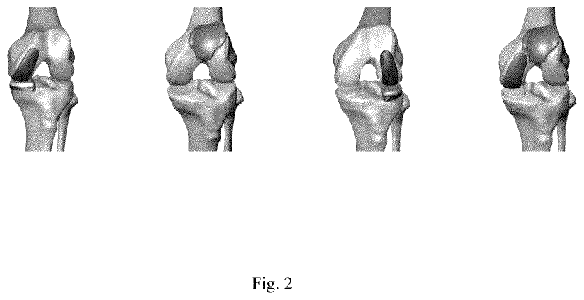

FIG. 2 is a schematic diagram showing typical partial knee arthroplasty procedures for replacing different compartments of the knee joint according to a specific embodiment of the present disclosure.

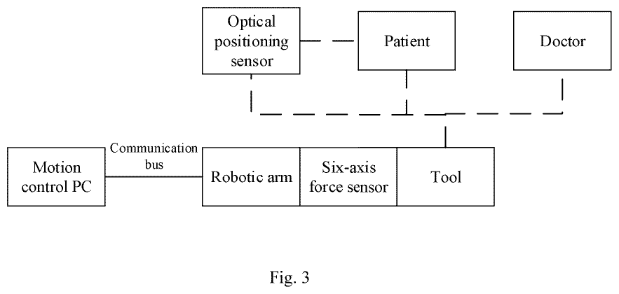

FIG. 3 is a schematic diagram showing the connection relationships among components in the complex spatial boundary control system for an orthopedic surgical robot according to a specific embodiment of the present disclosure.

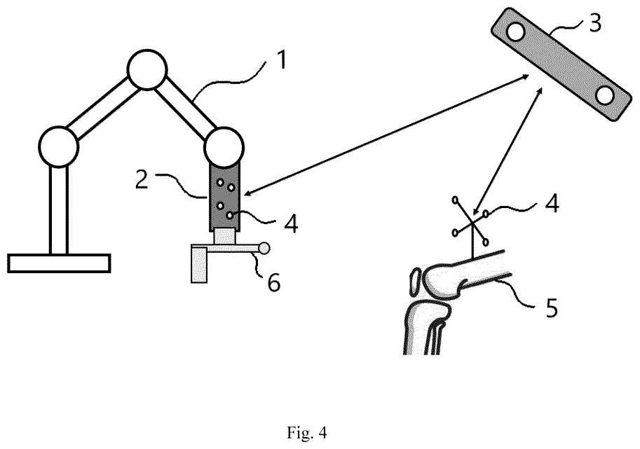

FIG. 4 is a schematic diagram showing the spatial hardware configuration of the system components in the complex spatial boundary control system for an orthopedic surgical robot according to a specific embodiment of the present disclosure.

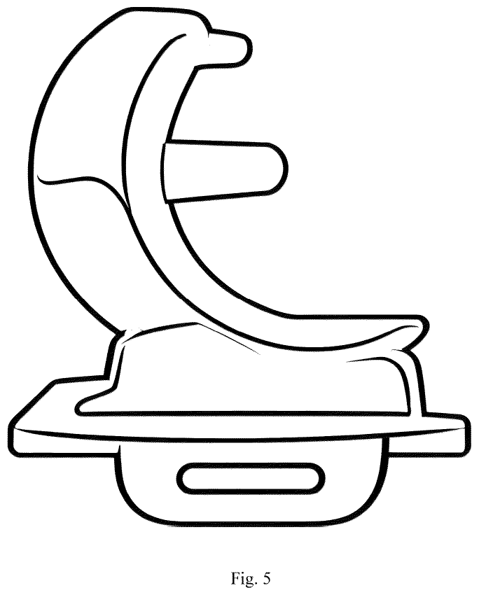

FIG. 5 is a schematic diagram showing typical medial/lateral compartment artificial prostheses according to a specific embodiment of the present disclosure.

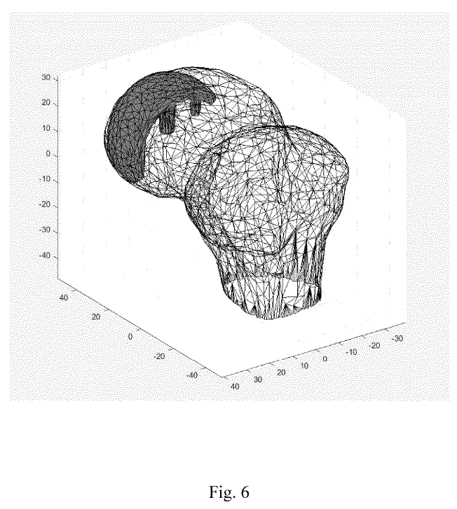

FIG. 6 is a schematic diagram showing the installation of typical medial/lateral compartment artificial prostheses according to a specific embodiment of the present disclosure.

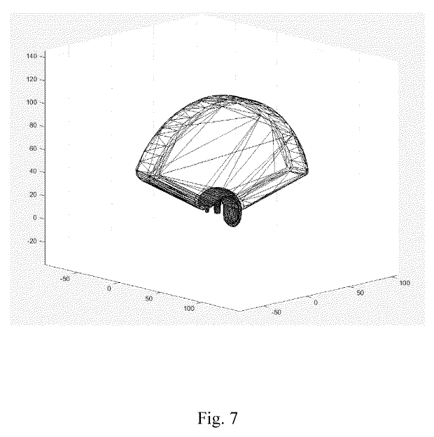

FIG. 7 is a schematic diagram showing the complex spatial boundary corresponding to a typical medial/lateral compartment artificial prosthesis according to a specific embodiment of the present disclosure.

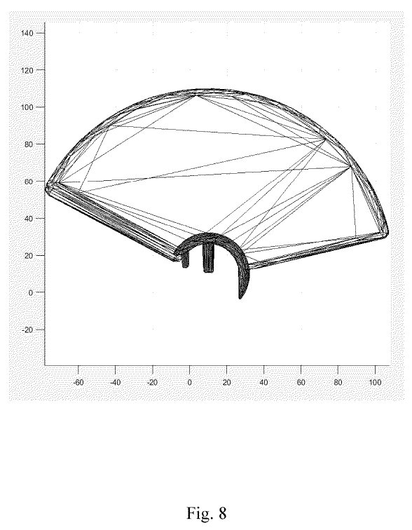

FIG. 8 is a schematic diagram of the same complex spatial boundary as in FIG. 7, viewed from a different angle, according to a specific embodiment of the present disclosure.

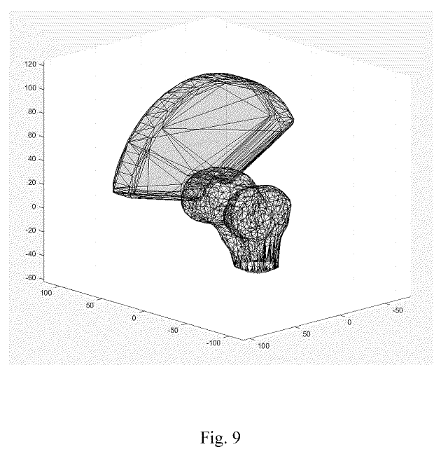

FIG. 9 is a schematic diagram showing the spatial relationship between the complex spatial boundary and the femur corresponding to a typical medial/lateral compartment artificial prosthesis according to a specific embodiment of the present disclosure.

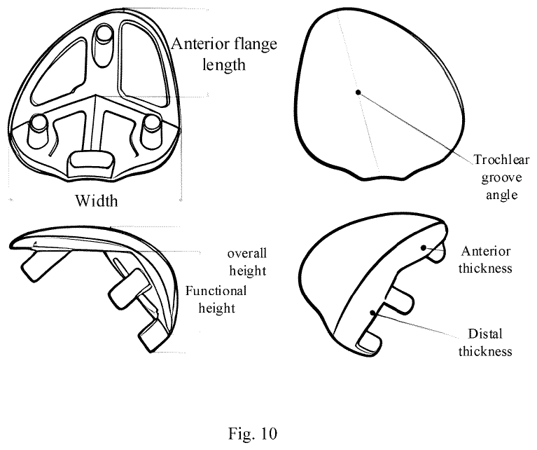

FIG. 10 is a schematic diagram showing a typical patellofemoral joint artificial prosthesis according to a specific embodiment of the present disclosure.

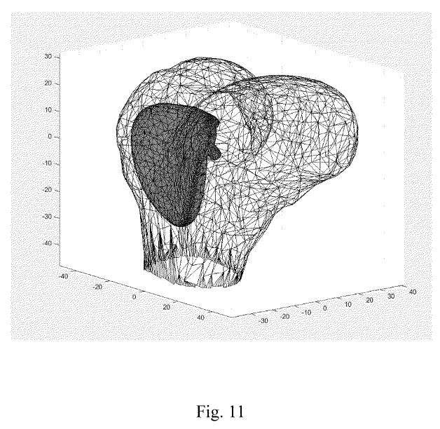

FIG. 11 is a schematic diagram showing the installation of a typical patellofemoral joint artificial prosthesis according to a specific embodiment of the present disclosure.

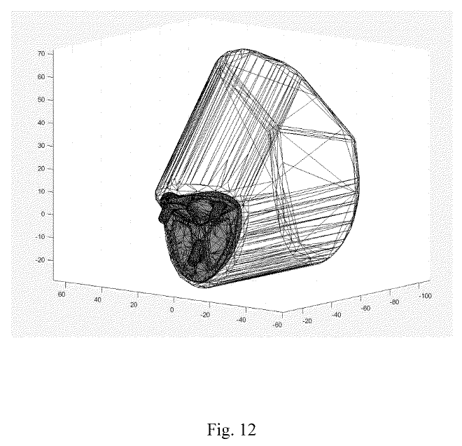

FIG. 12 is a schematic diagram showing the complex spatial boundary corresponding to a typical patellofemoral joint artificial prosthesis according to a specific embodiment of the present disclosure.

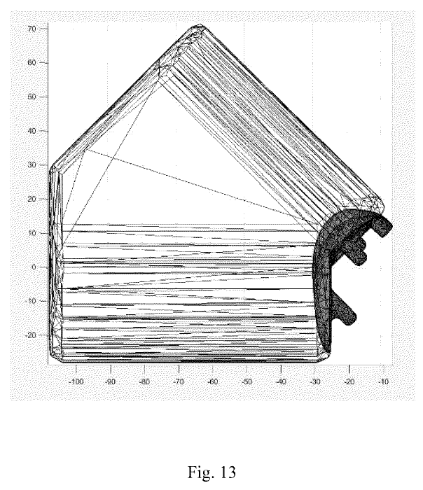

FIG. 13 is a schematic diagram of the same complex spatial boundary as in FIG. 12, viewed from another angle, according to a specific embodiment of the present disclosure.

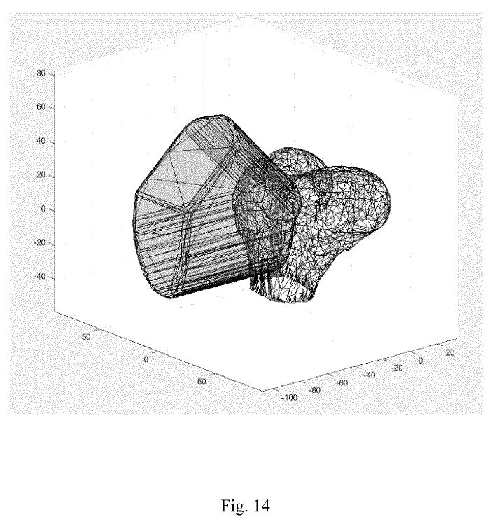

FIG. 14 is a schematic diagram showing the spatial relationship between the complex spatial boundary and the femur corresponding to a typical patellofemoral joint artificial prosthesis according to a specific embodiment of the present disclosure.

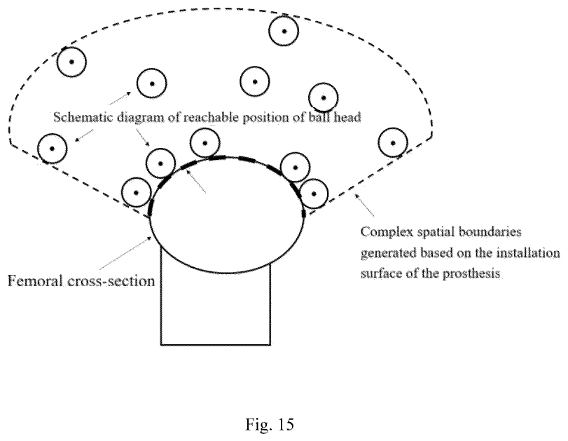

FIG. 15 is a schematic diagram showing the two-dimensional cross-sectional view of the complex spatial boundary corresponding to the femur according to a specific embodiment of the present disclosure.

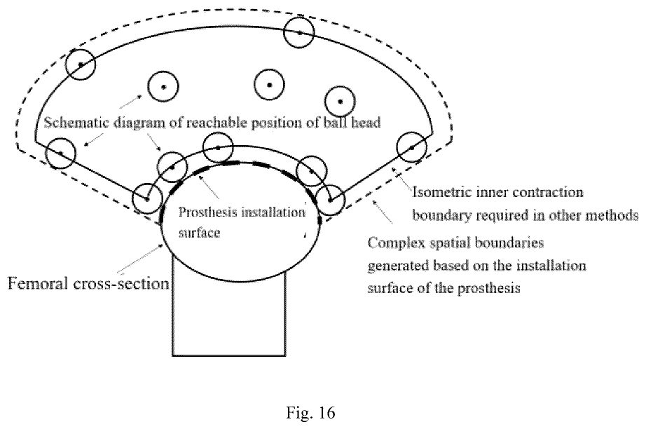

FIG. 16 is a schematic diagram showing the offset boundaries required by other existing methods in the prior art, according to a specific embodiment of the present disclosure.

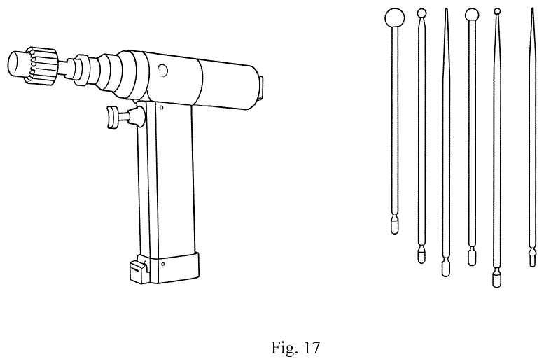

FIG. 17 is a schematic diagram showing the high-speed grinding drill and ball heads of different sizes and specifications according to a specific embodiment of the present disclosure.

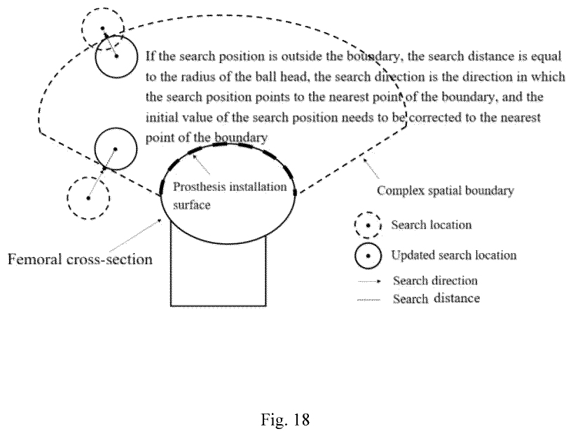

FIG. 18 is a schematic diagram illustrating the search principle when the search position is outside the boundary according to a specific embodiment of the present disclosure.

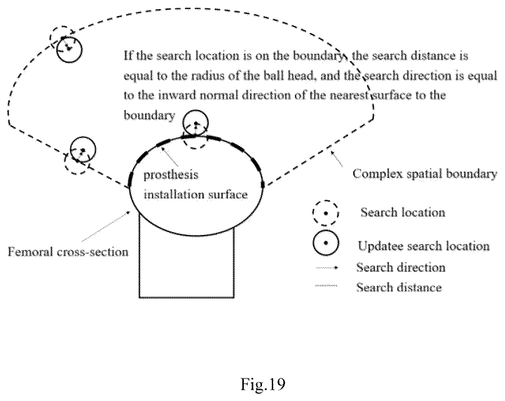

FIG. 19 is a schematic diagram illustrating the search principle when the search position is on the boundary according to a specific embodiment of the present disclosure.

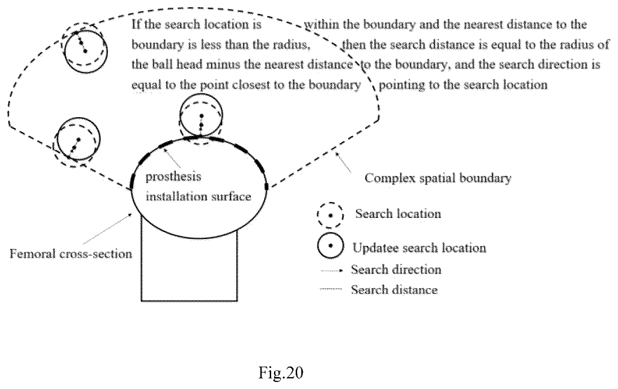

FIG. 20 is a schematic diagram illustrating the search principle when the search position is inside the boundary and further searching is required according to a specific embodiment of the present disclosure.

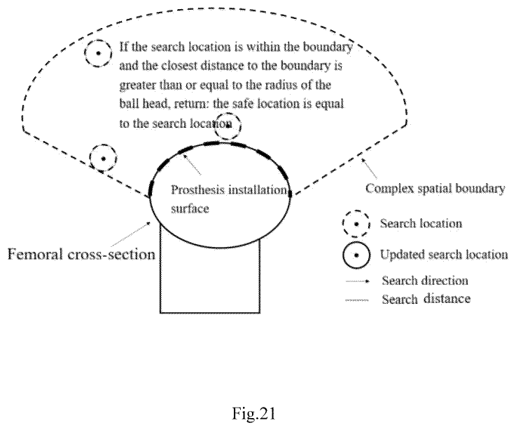

FIG. 21 is a schematic diagram illustrating the search principle when the search position is inside the boundary and no further searching is required according to a specific embodiment of the present disclosure.

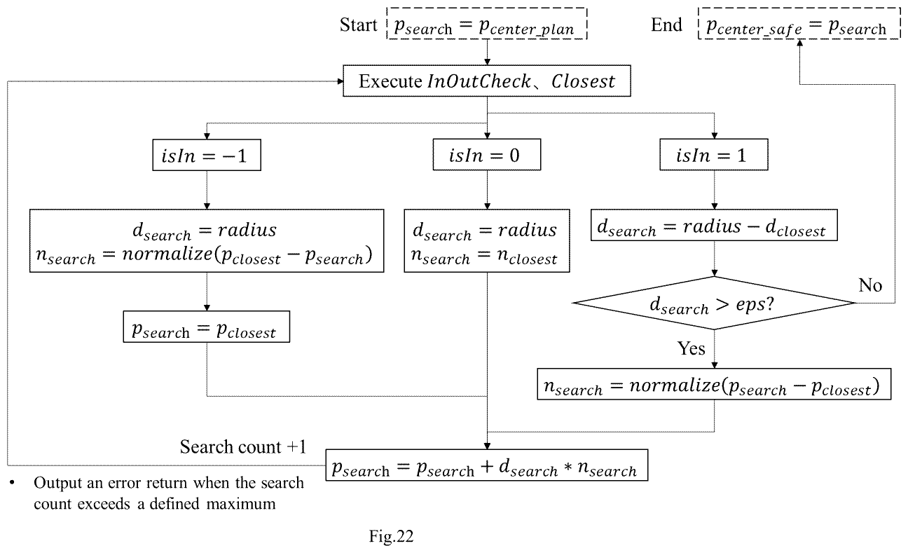

FIG. 22 is a schematic flowchart showing the algorithmic process of the complex spatial boundary control method for a surgical robot according to a specific embodiment of the present disclosure.

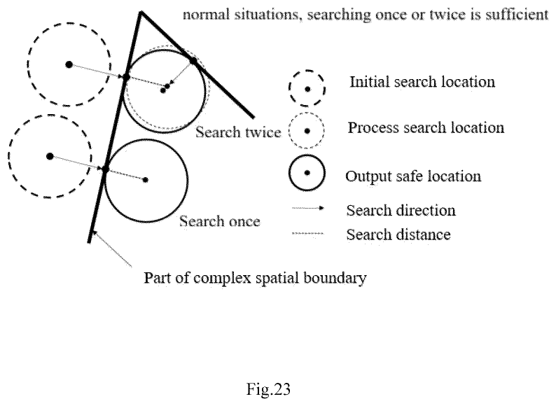

FIG. 23 is a schematic two-dimensional cross-sectional view illustrating a typical (normal) search process according to a specific embodiment of the present disclosure.

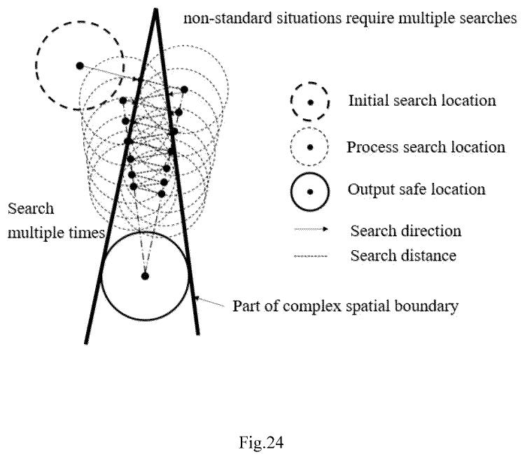

FIG. 24 is a schematic two-dimensional cross-sectional view illustrating an atypical (non-standard) search process according to a specific embodiment of the present disclosure.

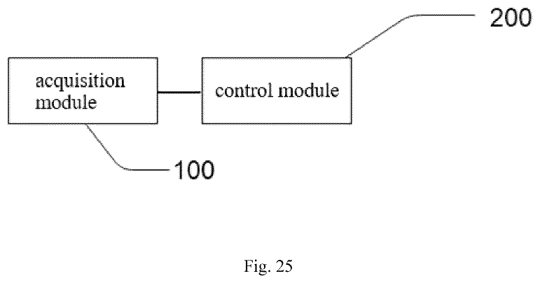

FIG. 25 is a schematic diagram showing the system architecture of the complex spatial boundary control system for an orthopedic surgical robot according to a specific embodiment of the present disclosure.

In the above drawings, the following reference numerals are used:
1: robotic arm;
2: six-axis force sensor (six-degree-of-freedom force sensor);
3: optical camera;
4: optical positioning sensor;
5: patient bone;
6: tool;
100: acquisition module;
200: control module.

### DETAILED DESCRIPTION OF EMBODIMENTS

To enable those skilled in the art to better understand the technical solutions of the present disclosure, the following provides a clear and complete description of the embodiments of the disclosure in conjunction with the accompanying drawings. It should be understood that, based on the embodiments in this application, other equivalent embodiments that do not require inventive effort by those of ordinary skill in the art shall also fall within the protection scope of the present application. In addition, in the embodiments mentioned below, directional terms such as “upper,” “lower,” “left,” and “right” refer only to the directions shown in the accompanying drawings. These directional terms are used for illustration and not for limitation of the disclosure.

The embodiments are further described in conjunction with the drawings below.

Please refer to FIGS. 1-25. The present disclosure provides a complex spatial boundary control method for an orthopedic surgical robot, comprising the following steps:

S100: obtaining a target desired position of a center of a target grinding drill ball head mounted at an operating end of the robot, complex spatial boundary data, and a radius of the target grinding drill ball head. The complex spatial boundary data is defined as a closed manifold polyhedron enveloped by triangular facets.

It should be noted that FIG. 2 illustrates the typical partial knee arthroplasty procedures for replacing different compartments of the knee joint. From left to right, these include: medial compartment replacement, patellofemoral joint replacement, lateral compartment replacement, and combined medial-patellofemoral joint replacement. Other combinations, such as medial-lateral compartment replacement, are also possible. All of these collectively fall under partial knee arthroplasty (PKA).

It should also be noted that the complex spatial boundary control method proposed by the present disclosure has a system composition as shown in FIGS. 3 and 4, including a motion control PC, a robotic arm 1, a six-axis force sensor 2, a tool 6 (high-speed grinding drill), an optical positioning sensor 4, and an optical camera 3. As shown in FIG. 4, the robotic arm 1 is equipped at its distal end with a six-axis force sensor 2. The force-sensing end of the sensor is fixedly connected to the high-speed grinding drill. During the surgery, the surgeon performs direct hand-held interactive operation with the high-speed grinding drill. During surgery, the surgeon performs direct hand-held interactive operation with the high-speed grinding drill. The motion control PC receives force data from the six-axis force sensor, which senses the interaction force applied by the surgeon's hand on the grinding drill. Based on the magnitude and direction of this interaction force, the motion control PC performs force-based motion control of the robotic arm, enabling the high-speed grinding drill to move interactively and controllably within the predefined complex spatial boundary. The optical positioning sensor is responsible for real-time detection of the relative spatial relationship between the patient's bone 5 and the tool 6, allowing the motion control PC to continuously calculate and update the pose (position and orientation) of the complex spatial boundary in real time.

Specifically, the “complex spatial boundary” is defined as a closed manifold polyhedron enveloped by triangular facets, which satisfies the following conditions:
Each face of the polyhedron is a triangle;
Each triangle shares at least one vertex or one edge with an adjacent triangle;
No two triangles intersect or overlap; and
The polyhedron is a manifold, meaning that each point on it has a neighborhood that can be represented by a coordinate system in Euclidean space and satisfies the local Euclidean property.

This definition can be further explained as follows: A closed manifold polyhedron enveloped by triangular facets is a mesh structure composed of triangles that are continuously connected to one another without any discontinuities. Each triangle, referred to as a triangular facet, has three vertices and three edges. The collection of these triangles forms a seamless polyhedral surface without any overlap or intersection. At the same time, the polyhedron must satisfy the definition of a manifold, meaning that, locally, it behaves as a smooth Euclidean surface that is continuous and differentiable throughout.

A closed manifold polyhedron formed by triangular facets is an important data structure widely used in computer graphics and computer-aided design (CAD). When representing complex geometrical shapes, the use of a triangular-facet set is a common and effective approach for describing both the shape and surface features of a three-dimensional object. The flexibility of triangular facets allows them to be combined in various ways to approximate complex geometries. By increasing or decreasing the number of triangles, or by adjusting their size, shape, and position, the approximation of the original geometry can be made. This adjustability allows us to perform precise or coarser approximations as required.

This adjustability allows accurate representation of both global forms and local features. Triangular facets can also represent local details with high curvature or complex shape, since smaller triangles can better preserve fine geometric features in those regions. By increasing the triangle density in areas requiring higher precision, one can achieve a more accurate local approximation of the geometry. This data structure is also fundamental to many numerical computation and simulation methods, and may be considered common general knowledge in the field.

In the present disclosure, the complex spatial boundary defined by a closed manifold polyhedron enveloped by triangular facets will hereinafter be referred to simply as the “complex spatial boundary.”

When used for motion control of a robotic arm, one or more faces of the given complex spatial boundary are typically generated to conform to the geometry of the artificial prosthesis in contact with the bone surface. The robotic arm controls the high-speed grinding drill such that the spherical surface of the drill's ball head moves in close conformity with these boundary surfaces to perform bone surface preparation. Several examples of artificial prostheses and their corresponding complex spatial boundaries are shown in FIGS. 5-14. It should be understood that the diagrams in FIGS. 5-14 only illustrate optional configurations of the complex spatial boundary and are not limiting. The complex spatial boundary control method proposed herein can be applied to bone surface preparation for any prosthesis geometry, provided that a closed manifold polyhedron enveloped by triangular facets can be generated to match the surface of the prosthesis that contacts the bone.

FIG. 15 illustrates, using a two-dimensional cross-section of the femur, the definition of the complex spatial boundary and the reachable positions of the high-speed grinding drill ball head. In conventional boundary control methods such as those disclosed in CN116712169A and CN118106975B, it is necessary—during actual application—to generate equidistant inward-shifted (offset) boundary models based on the radius of the tool's ball head (The offset distance is set according to the radius of the ball head used), and generate multiple actual compensation control models for different grinding radius sizes, and then perform actual grinding to compensate for position (as shown by the solid fan-shaped curves in FIG. 16). The above control methods are not applicable to high-speed grinding drill ball heads with radius sizes that have not been pre compensated by the model. In contrast, this method does not require the pre generation of multiple equidistant inner contraction boundary control models, nor does it require the storage, management, or scheduling of these models. Instead, it dynamically compensates for the radius of the high-speed drilling ball head currently in use through control methods. For various ball heads with different radius sizes, a unique complex spatial boundary generated according to the prosthesis installation surface (as shown by the fan-shaped dashed line in FIGS. 15 and 16) is used for control, which has better universality.

Specifically, FIG. 17 illustrates the high-speed grinding drill and ball heads of different sizes and specifications. By controlling the center position of the ball head via the robotic arm, the grinding region can be precisely manipulated, thereby achieving accurate bone surface preparation.

It can be understood that the core concept of the present disclosure is as follows: Based on the tool position planned by the surgeon's applied force, the safety boundary of the surgical area, and the radius of the high-speed grinding drill ball head being used, the system computes a safe tool position and transmits it to the robotic arm. The robotic arm then drives the surgical tool accordingly, enabling precise and safe bone surface preparation.

It should be noted that, before step S100, the method further comprises:

Pre-setting, in the control module 200, the first preset threshold, second preset threshold, third preset threshold, and fourth preset threshold.

It can be understood that the first preset threshold, second preset threshold, third preset threshold, and fourth preset threshold can be configured according to the actual requirements of the user of the present disclosure. The disclosure imposes no specific limitation on the numerical values of the first preset threshold, second preset threshold, third preset threshold, and fourth preset thresholds, so long as they are suitable for implementing the complex spatial boundary control method described herein.

Preferably, since the radius of the grinding drill ball head generally ranges from 0.75 mm to 6 mm, the following parameter settings are adopted based on extensive experimental testing by the inventors: The first preset threshold is preferably set to 0.001×the radius of the target grinding drill ball head, or approximately 0.00075 mm; the second preset threshold is preferably set to 100 iterations; the third preset threshold is preferably set to 0.01×the radius of the target grinding drill ball head, or approximately 0.06 mm; and the fourth preset threshold is also preferably set to 100 iterations. These parameter settings have been determined through extensive testing and have proven to effectively realize the complex spatial boundary control method proposed by the present disclosure.

S200: determining whether the target desired position is located outside the complex spatial boundary to obtain a judgment result; based on the judgment result, determining whether to correct the target desired position according to the radius of the target grinding drill ball head, to obtain a safe desired position of the center of the target grinding drill ball head.

Specifically, determining whether to correct the target desired position according to the radius of the target grinding drill ball head to obtain the safe desired position of the ball head center comprises:
When the target desired position is inside the complex spatial boundary, calculating the minimum distance from the target desired position to the complex spatial boundary and determining whether the minimum distance is greater than or equal to the radius of the target grinding drill ball head. According to this determination, deciding whether to correct the target desired position according to the ball head radius to obtain the safe desired position of the ball head center.

If the minimum distance is greater than or equal to the radius of the target grinding drill ball head, the target desired position is directly taken as the safe desired position of the ball head center.

If the minimum distance is less than the radius of the target grinding drill ball head, the target desired position is corrected according to the ball head radius to obtain the safe desired position of the ball head center.

Specifically, correcting the target desired position according to the radius of the target grinding drill ball head to obtain the safe desired position of the ball head center comprises:

Taking the target desired position as the initial search position, selecting the nearest point from the initial search position to the complex spatial boundary and defining it as the initial nearest point; calculating the distance from the initial search position to the initial nearest point as the target distance; calculating the difference between the radius of the target grinding drill ball head and the target distance as the distance to be corrected; taking the direction from the initial search position toward the initial nearest point as the initial search direction, and moving the initial search position along the initial search direction by the distance to be corrected to obtain the safe desired position.

Specifically, determining whether the target desired position is located outside the complex spatial boundary and, based on the judgment result, determining whether to correct the target desired position according to the radius of the target grinding drill ball head to obtain the safe desired position of the ball head center comprises:
When the target desired position is on the complex spatial boundary, correcting the target desired position according to the radius of the target grinding drill ball head to obtain the safe desired position of the ball head center.

Specifically, correcting the target desired position according to the radius of the target grinding drill ball head to obtain the safe desired position of the ball head center comprises:

Taking the target desired position as the initial search position, using the radius of the target grinding drill ball head as the distance to be corrected; taking the initial search position as the foot of the perpendicular, constructing a perpendicular line from the initial search position to the boundary on which it lies; taking the direction of the ray formed by the foot of the perpendicular and the perpendicular line as the initial search direction, and moving the initial search position along the initial search direction by the distance to be corrected to obtain the safe desired position.

Specifically, determining whether the target desired position is located outside the complex spatial boundary and, based on the judgment result, determining whether to correct the target desired position according to the radius of the target grinding drill ball head to obtain the safe desired position of the ball head center comprises:
When the target desired position is outside the complex spatial boundary, correcting the target desired position according to the radius of the target grinding drill ball head to obtain the safe desired position of the ball head center.

Specifically, correcting the target desired position according to the radius of the target grinding drill ball head to obtain the safe desired position of the ball head center comprises:

Selecting the nearest point from the target desired position to the complex spatial boundary and taking this nearest point as the initial search position; taking the radius of the target grinding drill ball head as the distance to be corrected; taking the initial search position as the foot of the perpendicular, constructing a perpendicular line from the initial search position to the boundary on which it lies; taking the direction of the ray formed by the foot of the perpendicular and the perpendicular line as the initial search direction, and moving the initial search position along the initial search direction by the distance to be corrected to obtain the safe desired position.

Specifically, moving the initial search position along the initial search direction by the distance to be corrected to obtain the safe desired position further comprises:

After moving the initial search position along the initial search direction by the distance to be corrected, a candidate desired position (position to be verified) is obtained. The minimum distance from the position to be verified to the complex spatial boundary is then calculated, and it is determined whether this minimum distance is greater than or equal to the radius of the target grinding drill ball head.

If not, the above correction process is repeated for the position to be verified, until the corrected position to be verified lies inside the complex spatial boundary and, after recalculating, the minimum distance from the expected position to be verified to the complex spatial boundary is greater than or equal to the radius of the target grinding drill ball head. The corrected position to be verified is then taken as the safe desired position.

If yes, the expected position to be verified is directly taken as the safe desired position.

Specifically, the method further comprises:

When the minimum distance from the corrected position to be verified to the complex spatial boundary is greater than or equal to the radius of the target grinding drill ball head, determining whether the difference between this minimum distance and the radius of the target grinding drill ball head is less than or equal to a first preset threshold. If yes, the corrected position to be verified is taken as the safe desired position.

If no, the above correction process is repeated for the position to be verified until the difference between the minimum distance and the radius of the target grinding drill ball head is less than or equal to the first preset threshold, and the position to be verified at that time is taken as the safe desired position.

Specifically, the method further comprises:

Recording the number of corrections, and determining whether the number of corrections is greater than or equal to a second preset threshold, and whether a safe desired position has been found. Based on these determinations, the system decides whether the correction of the target desired position is abnormal.

If the number of corrections is greater than or equal to the second preset threshold and a safe desired position has not been found, the correction of the target desired position is determined to be abnormal.

If the number of corrections is less than the second preset threshold and a safe desired position has been found, the correction of the target desired position is determined to be normal.

If the number of corrections is less than the second preset threshold and a safe desired position has not been found, the correction of the target desired position is determined to be normal, and the correction process continues.

Specifically, the method further comprises:

When the number of corrections is greater than or equal to the second preset threshold and no safe desired position has been found, determining whether the difference between the minimum distance from the current position to be verified to the complex spatial boundary and the radius of the target grinding drill ball head is less than or equal to a third preset threshold. If yes, the current position to be verified is taken as the safe desired position. If no, the above correction process is repeated for the current position to be verified, and the correction count is re-recorded, until the difference between the minimum distance and the radius of the target grinding drill ball head is less than or equal to the third preset threshold. The corrected position to be verified at that time is then taken as the safe desired position.

Specifically, the method further comprises:

Determining whether the re-recorded correction count is greater than or equal to a fourth preset threshold, and whether a safe desired position has been found. Based on these determinations, deciding whether the correction of the target desired position is abnormal.

If the re-recorded correction count is greater than or equal to the fourth preset threshold and no safe desired position has been found, the correction of the target desired position is determined to be abnormal.

If the re-recorded correction count is less than the fourth preset threshold and a safe desired position has been found, the correction of the target desired position is determined to be normal.

If the re-recorded correction count is less than the fourth preset threshold and a safe desired position has not been found, the correction of the target desired position is determined to be normal, and the correction process continues.

It should be noted that, when the correction of the target desired position is determined to be abnormal, the system outputs an alarm signal regarding the abnormal correction of the target desired position and terminates the correction process.

When the correction of the target desired position is determined to be normal, the system outputs a notification signal indicating that the correction is normal.

It can be understood that complex boundary data may have artificial design problems due to its own structure, such as holes, discontinuities, incompleteness on the surface of complex space boundaries, or self-intersection of triangular facets inside complex space boundaries, which do not conform to the definition of closed manifold polyhedra that do not conform to the envelope of triangular facets. In this case, even if calibration continues, it may not be possible to find the safe expected position. The present disclosure intelligently monitors and alarms for this situation. Through the above scheme, when the calibration times are large, the threshold can be intelligently adjusted, and whether the safe expected position is found again can be judged. If not found, calibration can continue and the second calibration times can be judged, making the present disclosure more reliable in judging abnormal corrections. First, high-precision is used. Determine the threshold, If no results are found after many attempts, search again with slightly lower accuracy. This avoids using a single threshold and a fixed number of iterations to search for results, allowing the present disclosure to quickly search for the desired safe location with slightly reduced accuracy, further improving the intelligence, availability, safety, reliability, and system robustness of the present disclosure.

S300: controlling motion of the operating end of the robot according to the safe desired position.

Specifically, the following example is provided to further explain the principle of the present disclosure:

To generate a safe position located within the complex spatial boundary, the disclosure proposes the following complex spatial boundary control method. The central idea is as follows: First, using the desired position of the ball head center of the high-speed grinding drill, determine the positional relationship between this desired position and the complex spatial boundary. If the desired position lies within the boundary and the minimum distance from the desired position to the boundary is greater than the ball head radius, the desired position is directly taken as the safe position and output. Otherwise, different search and step-correction rules are applied in the direction toward the interior of the boundary, until a position is found whose minimum distance from the boundary is greater than or equal to the ball head radius. This position is then taken as the safe position and output.

It should be understood that the stepwise search process described above is conceptually identical to the correction process discussed previously; the two are equivalent descriptions of the same computational procedure.

If a closed manifold polyhedron enclosed by triangular facets is represented by a data structure, it generally includes a vertex coordinate set composed of the coordinate data of the polyhedron vertices and a triangular facet index set composed of the indices of the triangular facets. The triangular patch index set is generally a multi row, three column array composed of integers. The first, second, and third columns of each row represent the index values of the first, second, and third vertices of the triangular patch in the vertex coordinate set, used to determine the three vertices that make up a triangular patch. This data structure may be stored in files with extensions such as .stl or .obj. The complex spatial boundary data is represented as boundary, and its specific data structure or storage format is not limited by the present disclosure.

To determine whether a given point is inside, on, or outside the complex spatial boundary, any conventional method may be used, such as the ray-casting (ray-intersection) algorithm, or open-source algorithms. The specific algorithm is not limited by the disclosure. The pseudocode for determining the relative positional relationship isIn between the ball head center position and the complex spatial boundary based on the complex spatial boundary data “boundary” and the ball head center position p_{center} is as follows:

$$[ isIn ] = InOutCheck ( boundary , {p}_{center} )$$

Among them, the meaning of isIn is as follows:

$$isIn = \{ \begin{matrix}1 , & inside the complex spatial boundary \\ 0 , & on the complex spatial boundary \\ - 1 , & outside the complex spatial boundary\end{matrix}$$

Among them, InOutCheck is the name of the algorithm function that determines the position of a point within/on/outside the complex spatial boundary.

To determine the position of a point in each triangular surface of the complex space boundary, the nearest point position, nearest distance, and inward normal direction on the nearest triangular surface can also be determined using any conventional method, such as calendar traversal, octree search, etc., or implemented using open source software, and are not limited in the present disclosure. Based on the complex spatial boundary data boundary and the position of the ball head and center p-ucenter, the pseudocode for calculating the nearest point position p-loss, nearest distance d_{closest}, and inward normal direction d_{closest} on the triangular surface closest to the boundary is as follows:

$$[ {p}_{closet} , {d}_{closet} , {n}_{closest} ] = Closest ( boundary , {p}_{center} )$$

Among them, Closest is the name of the algorithm function for calculating the nearest point position p_{closest}, nearest distance d_{closest}, and inward normal n_{closest} on the triangular surface closest to the boundary at the center of the ball head.

Based on this, the pseudocode of the complete method is as follows:

Complex Spatial Boundary Control Method for Orthopedic Surgical Robot

```
  Input variables:
  Desired position of the high-speed grinding drill ball head
center p_{center}_{_{—}}ₚₗₐₙ(x, y, z), denoted as p_{center}_{_{—}}ₚₗₐₙ;
  Radius of the high-speed grinding drill ball head, denoted as radius;
  Complex spatial boundary, denoted as boundary.
  Intermediate variables:
  Search position p_{search}(x, y, z), denoted as p_{search};
  Search distance, denoted as d_{search};
  Search direction n_{search}(x, y, z) denoted as n_{search}.
  Output variable:
  Safe position of the high-speed grinding drill ball head center, denoted as
p_{center}_{_{—}}_{safe}(x, y, z).
  Method steps:
  Initialization: Start and initialize variables p_{search} = p_{center}_{_{—}}ₚₗₐₙ. Proceed to
step 2.
  Based on p_{search} and boundary, execute the InOutCheck algorithm,
obtaining isIn flag. Execute the Closest algorithm, obtaining
p_{closest}, d_{closest}, n_{closest} vector. Proceed to step 3.
  If the isIn flag = −1, indicating that the current search position is outside the
boundary, set d_{search} = radius, n_{search} = normalize(p_{closest} − p_{search}), fix
p_{search} = p_{closest} and Proceed to step 6.
  If the isIn flag = 0, indicating that the current search position is on the
boundary, d_{search} = radius, n_{search} = n_{closest}, and proceed to step 6.
  If the isIn flag = 1, indicating that the current search position is inside the
boundary, compute d_{search} = radius − d_{closest}. If d_{search} > eps, set n_{search} =
normalize(p_{search} − p_{closest}), and proceed to step 6. If d_{search} ≤ eps, set
p_{center}_{_{—}}_{safe} = p_{search}, mark the search as successful, output variable and end.
Update search position: p_{search} = p_{search} + d_{search} * n_{search} , return to step 2.
```

In the above process, “normalize” means to normalize the vector to obtain the unit direction, where

$$normalize ( \vec{n} ) = \frac{\vec{n}}{\vert \vec{n} \vert}$$

eps is the minimum value, representing control accuracy, which can usually be set to 0.001 to 0.01 times the radius. In steps 3, 4, and 5, each jump to step 6 is to ensure that the updated search position meets the requirement of the high-speed grinding and drilling ball head's spherical surface being tangent to the nearest surface or point of the complex spatial boundary, thereby achieving a fast search to a safe position. The specific steps and corresponding search principle diagrams are shown in FIG. 18-21.

It should be understood that the above process can also be represented by FIG. 22, which shows the flow diagram of the algorithm. In engineering implementation, it is often necessary to impose a maximum limit on the number of search iterations to avoid an infinite loop.

An effective approach is to count the number of times step 6 is executed and to terminate the method with an error return when the count exceeds a defined maximum. Alternatively, the search count can be controlled by adjusting the numerical value (usually a slightly larger value to reduce the search count and achieve faster returns).

Since each search direction points toward the interior of the complex spatial boundary, and each search distance corresponds to the distance required for the ball head sphere to become tangential to the nearest boundary facet or the nearest boundary point, the proposed method can usually find a valid safe position in only one or two iterations under normal conditions (as illustrated in FIG. 23). The reason for the need for cyclic search and repeated judgment as shown above is that complex spatial boundaries have uncertain concavity and convexity, and may have shape feature angles with small angles. This may result in the search direction and distance obtained in one loop, which may exceed another boundary after performing a step search (as shown in FIG. 24). Therefore, it cannot be simply assumed that a safe position can be returned after obtaining the result variables of one or two searches and calculating the updated position step by step.

It should be noted that this method can not only be used for complex spatial boundary control in partial knee replacement surgery (PKA), but also extended to complex spatial boundary control in any surgical procedure. For example, when the complex spatial boundary is a sphere, it can be used for acetabular fossa grinding in THA surgery. When the complex space boundary is a cube, it can be used for planar osteotomy in TKA procedures, etc. This article uses partial knee replacement surgery (PKA) as an example only.

As shown in FIG. 25, the present disclosure further provides a complex spatial boundary control system for an orthopedic surgical robot. The system includes:
an acquisition module 100, configured to obtain the target desired position of the center of the ball head of the target grinding drill mounted at the operating end of the robot, the complex spatial boundary data, and the radius of the target grinding drill ball head; wherein the complex spatial boundary data is a closed manifold polyhedron enveloped by triangular facets;
and a control module 200, configured to determine whether the target desired position is located outside the complex spatial boundary, and, based on the judgment result, determine whether to correct the target desired position according to the radius of the target grinding drill ball head, thereby obtaining the safe desired position of the ball head center; and to control the motion of the robot's operating end according to the safe desired position.

In a preferred embodiment, the present application further provides an electronic device, which comprises:
a memory; and a processor, wherein the memory stores computer-readable instructions, and when the computer-readable instructions are executed by the processor, they implement the complex spatial boundary control method for an orthopedic surgical robot as described above. The computer device may broadly refer to a server, terminal, or any other electronic device having the necessary computing and/or processing capability. In one embodiment, the computer device may include a processor, memory, network interface, communication interface, and other components interconnected via a system bus. The processor of the computer device may provide the necessary computing, processing, and/or control capability.

The memory of the computer device may include non-volatile storage media and volatile memory. The non-volatile storage media may store an operating system, computer programs, et al. The volatile memory provides an environment for the operation of the operating system and computer programs stored in the non-volatile storage media. The network interface and communication interface of the computer device may be used to connect and communicate with external devices via a network. When executed by the processor, the computer program carries out the steps of the method of the present disclosure.

The present disclosure may also be implemented as a computer-readable storage medium having a computer program stored thereon. When executed by a processor, the computer program causes the steps of the method according to the embodiments of the present disclosure to be performed. In one embodiment, the computer program may be distributed across multiple computer devices or processors coupled through a network, such that the computer program is stored, accessed, and executed in a distributed manner by one or more computer devices or processors. A single method step or operation, or two or more method steps or operations, may be executed by a single computer device or processor, or by two or more computer devices or processors. One or more method steps or operations may be executed by one or more computer devices or processors, while one or more other method steps or operations may be executed by one or more other computer devices or processors. A single computer device or processor may execute one method step or operation, or may execute two or more method steps or operations.

Those skilled in the art will understand that the method steps of the present disclosure may be performed by hardware such as a computer device or processor under the control of a computer program, which may be stored in a non-transitory computer-readable storage medium. When executed, the computer program causes the steps of the disclosure to be performed. Depending on the circumstances, any reference in this specification to a memory, storage, database, or other medium may include non-volatile and/or volatile memory. Examples of non-volatile memory include Read-Only Memory (ROM), Programmable ROM (PROM), Erasable PROM (EPROM), Electrically Erasable PROM (EEPROM), flash memory, magnetic tape, floppy disk, magneto-optical data storage devices, optical data storage devices, hard disks, and solid-state drives (SSDs). Examples of volatile memory include Random Access Memory (RAM) and external cache memory.

The technical features described above may be combined arbitrarily. Although not all possible combinations of these technical features are described herein, any such combination shall be considered to fall within the scope of this specification, provided that no contradiction arises.

The specific embodiments of the present disclosure described above are not intended to limit the scope of protection of the present disclosure. Any modifications or equivalent substitutions made according to the technical concept of the present disclosure shall fall within the scope of protection defined by the claims of this application.

What is claimed is:

1. A complex spatial boundary control method for an orthopedic surgical robot, comprising:
S100: obtaining a target desired position of a center of a target grinding drill ball head mounted at an operating end of the robot, complex spatial boundary data, and a radius of the target grinding drill ball head; wherein the complex spatial boundary data is a closed manifold polyhedron enveloped by triangular facets;
S200: determining whether the target desired position is located outside the complex spatial boundary to obtain a judgment result, and based on the judgment result, determining whether to correct the target desired position according to the radius of the target grinding drill ball head, to obtain a safe desired position of the center of the target grinding drill ball head;
S300: controlling motion of the operating end of the robot according to the safe desired position.

2. The complex spatial boundary control method according to claim 1, wherein the determining, based on the judgment result, whether to correct the target desired position according to the radius of the target grinding drill ball head to obtain the safe desired position of the ball head center, comprises:
when the target desired position is located inside the complex spatial boundary, calculating a nearest distance from the target desired position to the complex spatial boundary, and determining whether the nearest distance is greater than or equal to the radius of the target grinding drill ball head; based on the determination, deciding whether to correct the target desired position according to the radius of the target grinding drill ball head to obtain the safe desired position of the ball head center, specifically:
when the nearest distance is greater than or equal to the radius of the target grinding drill ball head, directly using the target desired position as the safe desired position of the center of the target grinding drill ball head; and
when the nearest distance is less than the radius of the target grinding drill ball head, correcting the target desired position according to the radius of the target grinding drill ball head to obtain the safe desired position of the center of the target grinding drill ball head.

3. The complex spatial boundary control method according to claim 2, wherein the correcting the target desired position according to the radius of the target grinding drill ball head to obtain the safe desired position of the center of the target grinding drill ball head comprises:
using the target desired position as an initial search position, selecting a nearest point from the initial search position to the complex spatial boundary and defining the nearest point as an initial nearest point; calculating a distance from the initial search position to the initial nearest point as a target distance; calculating a difference between the radius of the target grinding drill ball head and the target distance as a distance to be corrected; taking a direction from the initial search position toward the initial nearest point as an initial search direction, and moving the initial search position along the initial direction by the distance to be corrected to obtain the safe desired position.

4. The complex spatial boundary control method according to claim 1, wherein the determining whether the target desired position is located outside the complex spatial boundary to obtain a judgment result, and based on the judgment result, determining whether to correct the target desired position according to the radius of the target grinding drill ball head to obtain the safe desired position of the center of the target grinding drill ball head, comprises:
when the target desired position is located on the complex spatial boundary, correcting the target desired position according to the radius of the target grinding drill ball head to obtain the safe desired position of the center of the target grinding drill ball head.

5. The complex spatial boundary control method according to claim 4, wherein the correcting the target desired position according to the radius of the target grinding drill ball head to obtain the safe desired position of the center of the target grinding drill ball head comprises:
using the target desired position as an initial search position, taking the radius of the target grinding drill ball head as a distance to be corrected; taking the initial search position as a foot of a perpendicular, constructing a perpendicular line from the initial search position to a boundary on which the initial search position lies; taking a direction of a ray formed by the foot of the perpendicular and the perpendicular line as an initial search direction; and moving the initial search position along the initial search direction by the distance to be corrected to obtain the safe desired position.

6. The complex spatial boundary control method according to claim 1, wherein the determining whether the target desired position is located outside the complex spatial boundary to obtain a judgment result and, based on the judgment result, determining whether to correct the target desired position according to the radius of the target grinding drill ball head to obtain the safe desired position of the center of the grinding drill ball head, comprises:
when the target desired position is located outside the complex spatial boundary, correcting the target desired position according to the radius of the target grinding drill ball head to obtain the safe desired position of the ball head center.

7. The complex spatial boundary control method according to claim 6, wherein the correcting the target desired position according to the radius of the target grinding drill ball head to obtain the safe desired position of the center of the target grinding drill comprises:
selecting a nearest point from the target desired position to the complex spatial boundary and taking the nearest point as an initial search position; taking the radius of the target grinding drill ball head as a distance to be corrected; taking the initial search position as a foot of a perpendicular, constructing a perpendicular line to a boundary on which the initial search position lies; taking a direction of a ray formed by the foot of the perpendicular and the perpendicular line as an initial search direction; and moving the initial search position along the initial search direction by the distance to be corrected to obtain the safe desired position.

8. The complex spatial boundary control method for an orthopedic surgical robot according to claim 3, wherein the moving the initial search position along the initial search direction by the distance to be corrected to obtain the safe desired position further comprises:
after moving the initial search position along the initial search direction by the distance to be corrected, obtaining a position to be verified; calculating a minimum distance from the position to be verified to the complex spatial boundary; and determining whether the minimum distance is greater than or equal to the radius of the target grinding drill ball head;
when the minimum distance is less than the radius of the target grinding drill ball head, repeating correction process on the position to be verified until the corrected position to be verified lies inside the complex spatial boundary and, after calculating the minimum distance from the corrected position to be verified to the complex spatial boundary, the minimum distance is greater than or equal to the radius of the target grinding drill ball head, and taking the corrected position to be verified as the safe desired position; and
when the minimum distance is greater than or equal to the radius of the target grinding drill ball head, directly taking the position to be verified as the safe desired position.

9. The complex spatial boundary control method according to claim 8, further comprising:
when the minimum distance from the corrected position to be verified to the complex spatial boundary is greater than or equal to the radius of the target grinding drill ball head, determining whether a difference between the minimum distance and the radius of the target grinding drill ball head is less than or equal to a first preset threshold;
when the difference between the minimum distance and the radius of the target grinding drill ball head is less than or equal to a first preset threshold, taking the corrected position to be verified as the safe desired position;
when the difference between the minimum distance and the radius of the target grinding drill ball head is greater than a first preset threshold, repeating the correction process on the position to be verified until the difference between the minimum distance and the radius of the target grinding drill ball head is less than or equal to the first preset threshold, and taking the corrected position to be verified at that time as the safe desired position.

10. The complex spatial boundary control method according to claim 9, further comprising:
recording a number of corrections, determining whether the number of corrections is greater than or equal to a second preset threshold, and determining whether a safe desired position has been found; and based on these determinations, deciding whether the correction of the target desired position is abnormal;
when the number of corrections is greater than or equal to the second preset threshold and no safe desired position has been found, determining that the correction of the target desired position is abnormal;
when the number of corrections is less than the second preset threshold and a safe desired position has been found, determining that the correction of the target desired position is normal; and
when the number of corrections is less than the second preset threshold and no safe desired position has yet been found, determining that the correction of the target desired position is normal, and continuing the correction of the target desired position.

11. The complex spatial boundary control method according to claim 10, further comprising:
when the number of corrections is greater than or equal to the second preset threshold and no safe desired position has been found, determining whether the difference between the minimum distance from the current position to be verified to the complex spatial boundary and the radius of the target grinding drill ball head is less than or equal to a third preset threshold;
when the difference between the minimum distance from the current position to be verified to the complex spatial boundary and the radius of the target grinding drill ball head is less than or equal to a third preset threshold, taking the current position to be verified as the safe desired position;
when the difference between the minimum distance from the current position to be verified to the complex spatial boundary and the radius of the target grinding drill ball head is greater than a third preset threshold, repeating the above correction process on the current position to be verified, and re-recording the correction count, until the difference between the minimum distance and the radius of the target grinding drill ball head is less than or equal to the third preset threshold, and taking the corrected position to be verified at that time as the safe desired position.

12. The complex spatial boundary control method according to claim 11, further comprising:
determining whether a re-recorded number of corrections is greater than or equal to a fourth preset threshold, and determining whether a safe desired position has been found; and based on the judgment results, determining whether the correction of the target desired position is abnormal;
when the re-recorded number of corrections is greater than or equal to the fourth preset threshold and no safe desired position has been found, determining that the correction of the target desired position is abnormal;
when the re-recorded number of corrections is less than the fourth preset threshold and a safe desired position has been found, determining that the correction of the target desired position is normal; and
when the re-recorded number of corrections is less than the fourth preset threshold and no safe desired position has yet been found, determining that the correction of the target desired position is normal, and continuing the correction of the target desired position.

13. A complex spatial boundary control system for an orthopedic surgical robot, comprising:
an acquisition module, configured to obtain a target desired position of a center of a target grinding drill ball head mounted at an operating end of the robot, complex spatial boundary data, and a radius of the target grinding drill ball head; wherein the complex spatial boundary data is a closed manifold polyhedron enveloped by triangular facets; and
a control module, configured to determine whether the target desired position is located outside the complex spatial boundary to obtain a judgment result, and based on the judgment result, determine whether to correct the target desired position according to the radius of the target grinding drill ball head, to obtain the safe desired position of the center of the target grinding ball head; and to control motion of the operating end of the robot according to the safe desired position.

14. An electronic device, comprising:
a memory; and a processor, wherein computer-readable instructions stored in the memory are executed by the processor to implement the complex spatial boundary control method according to claim 1.
